(19) United States
(12) Patent Application Publication
(10) Pub. No.: US 2025/0210200 A1
(43) Pub. Date: Jun. 26, 2025

(54) METHOD FOR IDENTIFYING LIVER FIBROSIS

(71) Applicant: Thomas Anthony Beltran, OLYMPIA, WA (US)

(72) Inventor: Thomas Anthony Beltran, OLYMPIA, WA (US)

(73) Assignee: Thomas Anthony Beltran, OLYMPIA, WA (US)

(21) Appl. No.: 18/396,083

(22) Filed: Dec. 26, 2023

(51) Int. Cl. G16H 50/30; G01N 33/52; G01N 33/573; G16H 50/20

(52) U.S. Cl. CPC G16H 50/30; G01N 33/52; G01N 33/573; G16H 50/20; G01N 2333/9108; G01N 2333/91188; G01N 2800/085; G01N 2800/52

(57) ABSTRACT

Provided herein is a novel method for identifying individuals suffering from liver fibrosis. The methods disclosed herein employ a combination of specific biomarkers and patient demographic information. These biomarkers include Gamma Glutamyl Transferase (GGT), and Aspartate Aminotransferase (AST) or Alanine Aminotransferase (ALT), as well as patient-specific variables such as age, standing height, weight, and diabetes status. By combining these elements, the method aims to generate a diagnostic score, which is then compared to a predefined threshold to categorize the individual's stage of liver fibrosis. The stages of interest include significant fibrosis (METAVIR stages F2 to F4), advanced fibrosis (METAVIR stages F3 and F4), or cirrhosis (METAVIR stage F4). The method's strength lies in its ability to accurately assess liver fibrosis using readily obtainable data, facilitating extensive screening and contributing to the early detection of this condition.

### FIELD OF THE INVENTION

The present invention is related to the field of medical diagnostics. In particular, the invention focuses on the diagnosis and staging of hepatic (liver) fibrosis.

### BACKGROUND OF THE INVENTION

Non-alcoholic fatty liver disease (NAFLD) is a common cause of chronic liver dysfunction, affecting a quarter of adults in the U.S. (Hepatology 64:73-84 (2016)). Clinically, the spectrum of NAFLD ranges from non-alcoholic fatty liver (hepatic steatosis) to non-alcoholic steatohepatitis (NASH). Hepatic (liver) scarring (fibrosis) is directly linked to NASH and is a leading cause of cirrhosis in the United States (Hepatology 51:1820-1832 (2010); Liver international 41:78-82 (2021)).

Liver fibrosis results from the accumulation of extracellular matrix proteins and is common to many etiologies (Annu Rev Pathol 6:425-56 (2011)). Such diseases include but are not limited to chronic viral hepatitis B and C, autoimmune hepatitis, alcoholic liver disease, and drug-induced liver disease. If left untreated, liver fibrosis may progress resulting in cirrhosis, liver failure, or portal hypertension (Digestive diseases 33:492-7 (2015)). These end stage conditions often require liver transplantation in order to save the patient.

Liver fibrosis is commonly categorized using the meta-analysis of histological data in viral hepatitis (METAVIR) score (J gastroenterology and hepatology 32:548-57 (2017)). METAVIR stages are used to classify the severity of fibrosis seen on a liver biopsy sample. Under this paradigm, F0 equates to no fibrosis; F1 refers to portal fibrosis without septa; F2 refers to portal fibrosis with few septa; F3 refers to numerous septa without cirrhosis; and F4 refers to the presence of hepatic cirrhosis. However, more commonly METAVIR stages refer to a lack of or minimal fibrosis (F0/1), significant fibrosis (F2), advanced fibrosis (F3), or cirrhosis (F4) (Hepatology 24:289-93 (1996)).

Evaluation resulting in identification of METAVIR stage F0 or F1 does not typically require intervention. However, the presence of significant fibrosis (METAVIR stage F2 or greater) is widely accepted as an indication to treat an individual (Gut 49:11-21 (2001); Hepatology 39:1147-71 (2004); Gastroenterology 161:1657-69 (2021)). Whereas the presence of cirrhosis (METAVIR stage 4) may indicate assessing the individual for hepatocellular carcinoma or esophageal varices resulting diminished blood flow through the portal vein (Hepatology 23:10-97 (2023)).

Liver biopsy has historically been considered the gold standard for the definitive assessment of hepatic fibrosis, providing detailed information about the extent and severity of liver damage. However, this procedure is invasive and is associated with potential complications. The complication rate requiring hospitalization can be as high as approximately three percent. These complications may include bleeding, infection, and other adverse events, highlighting the need for alternative, less invasive methods for assessing hepatic fibrosis (Hepatology 49:1017-44 (2009)). Consequently, noninvasive methods such as biomarkers and elastography are commonly used as surrogate markers to estimate hepatic fibrosis, thus providing a safer and less costly strategy to identify the disease (WJG 20:16820 (2014); Annals of internal medicine 158:807-20 (2013)).

Vibration-controlled Transient Elastography (VCTE) represents a non-invasive method for assessing liver fibrosis by measuring liver stiffness. The technique involves the use of specialized equipment that generates controlled vibrations and measures the resulting shear wave propagation through liver tissue. The velocity of these waves correlates with the stiffness of the liver, providing valuable information about the extent of fibrosis. (Liver international 37:851-61 (2017); Annals of internal medicine 158:807-20 (2013)). While VCTE has proven efficacy in fibrosis evaluation, its widespread use is limited by the need for specialized equipment and expertise. The technology requires trained professionals to perform the procedure accurately and interpret the results correctly, making it less accessible in certain healthcare settings (United European gastroenterology journal 7:1113-23 (2019)). As a result, serological markers are typically utilized as a first-line screening measure both for their lower risk and cost (Current pharmaceutical design 24:4574-86 (2018)).

Commonly employed models used to estimate the presence of significant fibrosis include the aspartate aminotransferase (AST) to platelet ratio index (APRI), FIB-4 index, NAFLD fibrosis score (NFS), FORNS index, and BARD score. Although such models demonstrate high negative predictive value (NPV) for identifying individuals without significant fibrosis, they have poor positive predictive value (PPV) for identifying individuals with significant fibrosis. Thus, their strength lies in excluding rather than identifying or diagnosing significant fibrosis (J Hepatology 60:384-91 (2014); PLoS One 10: e0144425 (2015)). The diagnostic performance of non-invasive multiparametric models for liver assessment, while valuable, still has room for improvement, especially in the accurate identification of individuals with significant liver fibrosis. Liver fibrosis is a critical aspect of liver health assessment, as it represents the progression of liver damage and is a key factor in determining the severity of liver diseases.

### SUMMARY OF THE INVENTION

Provided herein are methods for identifying the presence of significant liver fibrosis in an individual. The method is accomplished using a formula to derive a score based on the individual's age, systolic blood pressure, standing height, weight, gamma glutamyl transferase (GGT), aspartate aminotransferase (AST) or alanine aminotransferase (ALT), and diabetes status (has diabetes or does not have diabetes).

This method is accomplished by obtaining a serum or plasma sample from the individual and determining the levels of GGT in international units per liter (IU/L) and either AST in units per liter (U/L) or ALT in U/L. Additionally, the individual should be assessed for systolic blood pressure (mmHg), standing height (cm), and weight (kg). The individual's body mass index (BMI) may be computed from the standing height and weight measurements using the equation:

$$BMI = ( weight in kg ) / {( 0.01 * standing height in cm )}^{2}$$

Finally the individual's age in years and diabetes status should be ascertained (1 if the person has diabetes, 0 if the person does not have diabetes). The values of the aforementioned markers and individuals characteristics are then used in a second equation to determine the end value (score).

Liver fibrosis is commonly scored on the 5-point METAVIR scale as follows: F0=no fibrosis, F1=portal fibrosis without septa; F2=portal fibrosis with few septa; F3=portal fibrosis with numerous septa without cirrhosis; and F4 refers to the presence of hepatic cirrhosis. Colloquially, the term “no significant fibrosis” refers to stages F0 and F1, the term “significant fibrosis” corresponds to stages F2, F3, and F4, and the term “advanced fibrosis” refers to stages F3 and F4 only. The term “cirrhosis” only refers to stage F4.

The term “score” as used herein represents a value of y, calculated using the following equation, henceforth referred to as equation 1:

$$y = {Lg}_{10} ( systolic blood pressure * BMI ) + ( 0.02 * GGT ) + ( 0.6 * ( AST or ALT ) * diabetes ) + ( 16 * ( weight / standing height ) ) + ( 5 * {Lg}_{10} ( age ) )$$

wherein systolic blood pressure is provided in mmHg; GGT is provided in IU/L; diabetes=1 if individual has a prior diagnosis of either type I or type II diabetes, diabetes=0 if the individual has no prior diagnosis of diabetes; AST is provided in IU/L; standing height is provided in cm; weight is provided in kg; and age is provided in years.

The term “coefficient” as used herein refers to the factors that each variable (i.e. systolic blood pressure, BMI, GGT, AST, diabetes, standing height, weight, and age) is multiplied or divided by in the aforementioned equation 1.

The numerical definitions of the coefficients in equation 1 can be varied and still produce a valid intermediate value. For example, the GGT coefficient may vary from 0.001 to 0.02; the AST coefficient may vary from 0.01 to 0.5; the (weight/standing height) coefficient may vary from 5 to 50; and the age coefficient may vary from 0.1 to 15. If units other than those previously mentioned are used, one would need to convert them into the appropriate units (i.e. if weight is available in pounds (lbs), convert lbs to kilograms (kg) by dividing the weight by 2.02462).

The term “about” as used herein in reference to numbers or quantitative measurements refer to the indicated value plus or minus 10%.

The score is compared with a cut-off value in order to determine the appropriate fibrosis classification. If the score is less than about 21.34 the individual is classified as having no significant fibrosis. If the individual's score is about 21.34 or greater but less than about 21.63, the individual should be classified as having significant fibrosis (stage F2-F4). A score equal to or greater than about 21.63 but less than about 22.84 indicates the individual should be classified as having advanced fibrosis (stage F3-F4). A score greater than or equal to about 22.84 indicates the individual should be classified as having cirrhosis.

In another aspect, a score of less than about 22.84 indicates an absence of cirrhosis. A score of less than about 21.63 indicates an absence of advanced fibrosis. A score of less than about 21.34 indicates an absence of significant fibrosis.

Also provided herein is a method of monitoring the evolution of liver fibrosis over time in an individual with liver disease. The method is accomplished by obtaining a baseline score for an individual and one or more additional scores based at some time point after the initial baseline score. Scores subsequent to the initial baseline score must be determined using new measurements of the component variables (i.e. systolic blood pressure, BMI, GGT, AST or ALT, diabetes, standing height, weight, and age). The extent of liver fibrosis as indicated by the first score is compared to the extent of liver fibrosis as indicated by subsequent scores. Furthermore, if subsequent scores are higher than the baseline score, this indicates progression of liver fibrosis. If however, there is a decrease in score from baseline to the subsequent score, this indicates a regression of liver fibrosis.

The term “therapy” as used herein refers to any manner of treatment of a disease or symptoms thereof. Therapy of liver fibrosis includes any accepted or experimental treatment. Therapy may include treatment or removal of the causal agent or treatment of the fibrosis with drug compounds or other therapeutic agents.

Monitoring of liver fibrosis therapy may be accomplished using the same method as stated above by comparing a score taken before treatment to a score during or after therapy is concluded.

The terms “efficacy” or “efficacious” as used herein refers to the ability of a drug, therapy or treatment to relieve symptoms or eliminate the disease. A treatment is said to have efficacy if certain positive outcomes, for example, a regression of extent of liver fibrosis, occur as a result of the treatment.

Invention methods may be used for a patient suffering from or screening one or more patients suspected of suffering from any disease involving liver fibrosis. In particular, the method of the invention can be performed for identifying liver fibrosis in patients suffering from, for example, hepatitis B, hepatitis C, alcoholism and alcohol abuse, alcoholic liver disease, hemochromatosis, metabolic disease, diabetes, obesity, autoimmune hepatitis, nonalcoholic fatty liver disease, alcoholic fatty liver, drug-induced liver disease, primary biliary cirrhosis, primary sclerosing cholangitis, a1-antitrypsin deficiency, Wilson disease, and chronic rejection or recurrent liver disease following liver transplantation.

Alcoholic liver disease encompasses a range of disorders, progressing from alcoholic fatty liver (i.e., steatosis) to alcoholic hepatitis and cirrhosis. These conditions exhibit distinct pathological features, and any or all of them may co-occur within the same patient. Alcoholic fatty liver is characterized by hepatocyte fat accumulation due to alcohol abuse, potentially leading to inflammation (steatohepatitis) and subsequent liver scarring (cirrhosis). Alcoholic hepatitis, which can occur independently or in conjunction with cirrhosis, is marked by liver cell necrosis and inflammation, with histological indicators including swollen hepatocytes and steatosis. Cirrhosis, the most severe form, arises from the replacement of damaged cells with connective tissue, resulting in liver scarring and eventual failure.

Nonalcoholic fatty liver disease (NAFLD), including metabolically associated NAFLD, predominantly affects overweight or glucose-intolerant individuals who do not excessively consume alcohol. NAFLD encompasses a spectrum of liver disorders, including simple fatty liver (steatosis), nonalcoholic steatohepatitis (NASH), and cirrhosis (irreversible, advanced liver scarring). All stages of NAFLD involve hepatocyte fat accumulation. In NASH, this accumulation is associated with varying degrees of inflammation (hepatitis) and liver scarring (fibrosis). Despite the absence of excessive alcohol consumption in individuals with NAFLD and NASH, histological analysis of liver biopsy samples reveals similarities to those from patients with alcohol-induced liver disease.

In a particular aspect, invention methods may be used to both monitor and estimate the fibrosis classification of patients diagnosed with or suspected of having fatty liver disease as a result of alcohol abuse or non-alcoholic fatty liver disease.

As used herein the term “primary care facility” means a facility that offers first-contact health care only. As used herein the term “secondary care” refers to services provided by medical specialists who generally do not have first contact with patients (e.g., cardiologist, urologists, dermatologists) such typically occurs in a local (or community) hospitals setting. As used herein, the term “tertiary care facility” means a facility that receives referrals from both primary and secondary care levels and usually offers tests, treatments, and procedures that are not available elsewhere.

The patient population to which the invention pertains is preferably patients receiving tertiary care such as in a tertiary care setting, although patients receiving primary and secondary care also can be evaluated using the invention methods. Thus, in a preferred embodiment, the individual to be tested is receiving tertiary medical care. In further embodiments, the individual to be tested is receiving primary or secondary medical care.

The secondary patient population to which the invention pertains is preferably patients in a primary care setting. In this aspect, the invention may be employed as a general screening tool to identify patients of concern among those otherwise not suspected of having fibrosis.

The term “disease” as used herein refers to an interruption, cessation, or disorder of body functions, systems, or organs and is characterized usually by a recognized etiologic agent(s), an identifiable group of signs and symptoms, or consistent anatomical alterations.

The term “symptom” as used herein refers to an indication or sign that a person has a disease and include changes from normal anatomical structure or bodily function.

As used herein, the terms “level” or “concentration” of a marker are used interchangeably and refer to the relative or absolute amount or activity of the marker per unit volume by any direct or indirect measurement. One of skill in the art would recognize that any assay useful for determining the level of a marker may be used in invention methods, provided such methods produce a level comparable to that obtained with the preferred methods described herein.

In another aspect, provided herein is a system for diagnosing the presence of liver fibrosis in an individual. This system comprises an input device in data communication with a processor, which is in data communication with an output device.

The input device is used for entry of data including levels of systolic blood pressure, GGT, AST, weight, standing height, diabetes status, and age as determined from a sample from the individual. Data may be entered manually by an operator of the system using a keyboard or keypad. Alternatively, data may be entered electronically, when the input device is a cable in data communication with a computer, a network, a server, or analytical instrument.

The processor comprises software for computing a score, y to diagnose liver fibrosis. The processor computes the score, y, using an algorithm, wherein the algorithm is:

$$y = {Lg}_{10} ( systolic blood pressure * BMI ) + ( 0.02 * GGT ) + ( 0.6 * AST or ALT * diabetes ) + ( 16 * ( weight / standing height ) ) + ( 5 * {Lg}_{10} ( age ) )$$

wherein,
systolic blood pressure is in mmHg,
BMI=(weight in kg)/(standing height in m)²,
Gamma Glutamyl Transferase (GGT) is in (IU/L),
diabetes=1 if the individual has diabetes, diabetes=0 if the individual does not have diabetes,
Aspartate Aminotransferase (AST) is in (U/L),
weight is in kilograms (kg),
standing height is in centimeters (cm),
age is in years.

The processor further compares the score to a cutoff value to diagnose the presence of liver fibrosis. If the score is less than about 21.34 the individual is classified as having no significant fibrosis. If the individual's score is about 21.34 or greater but less than about 21.63, the individual should be classified as having significant fibrosis (stage F2-F4). A score equal to or greater than about 21.63 but less than about 22.84 indicates the individual should be classified as having advanced fibrosis (stage F3-F4). A score greater than or equal to about 22.84 indicates the individual should be classified as having cirrhosis. A score less than about 21.34 is indicative of an absence of cirrhosis. The above numbers are subject to 5% variation.

The data output device, in data communication with the processor, receives the diagnosis from the processor and provides the diagnosis to the system operator. The output device can consist of, for example, a video display monitor or a printer.

As used herein, the term “specificity” denotes the likelihood that a diagnostic method of the invention produces a negative result when the sample is devoid of positivity, such as in the case of significant fibrosis (i.e., stage F2-F4). Specificity is computed by dividing the number of true negative results by the sum of true negatives and false positives. Specificity serves as a fundamental gauge of the method's effectiveness in excluding individuals who do not exhibit a particular disease or symptom, such as significant fibrosis.

As used herein, the term “sensitivity” pertains to the attribute of a diagnostic test that gauges its capacity to identify a disease (or symptom) accurately when it genuinely exists. Consequently, sensitivity denotes the fraction of all afflicted individuals who receive a positive test result, calculated by dividing the number of true positives by the sum of true positives and false negatives.

As used herein, the term “negative predictive value,” herein referred to as “NPV,” is defined as the probability that an individual diagnosed as lacking fibrosis indeed does not have the disease. The calculation of negative predictive value involves dividing the number of true negatives by the sum of true negatives and false negatives. The determination of negative predictive value is contingent upon the characteristics of the diagnostic method utilized and the prevalence of fibrosis within the analyzed population.

As used herein, the term “positive predictive value,” herein referred to as “PPV,” is defined as the likelihood that an individual identified with fibrosis genuinely manifests the disease or symptom. The calculation of positive predictive value involves dividing the number of true positives by the sum of true positives and false positives. The determination of positive predictive value is contingent upon the attributes of the diagnostic methodology employed and the prevalence of fibrosis within the scrutinized population.

As used herein, the term “diagnostic odds ratio”, herein referred to as “DOR” serves as a comprehensive measure that combines sensitivity and specificity into a single statistic to assess the discriminatory power of a diagnostic method. The DOR represents the ratio of the odds of a positive test result in individuals with the disease to the odds of a positive test result in individuals without the disease. It is calculated as the product of the cross-products of the true positives and true negatives divided by the product of the cross-products of false positives and false negatives. A higher DOR indicates better discriminatory performance, capturing both the ability to identify true positives and the ability to exclude false positives. The DOR is particularly valuable in evaluating the overall diagnostic efficacy of a method, providing a more nuanced understanding beyond individual measures like sensitivity and specificity. The DOR is less affected by disease prevalence than simple PPV or NPV and thus is more appropriate for assessing models of low incidence diseases such as liver fibrosis (Int J cadriovasc sci 29:218-22 (2016)).

As used herein, the term “accuracy” denotes the comprehensive alignment between the diagnostic method and the disease state. The calculation of accuracy involves the summation of true positives and true negatives, divided by the total number of sample results, with its determination impacted by the prevalence of fibrosis within the analyzed population.

As used herein, the term “Predictive Liver Assessment” or “PLA” refers to the score generated by equation 1.

### BRIEF DESCRIPTION OF FIGURES

FIG. 1A-C: Receiver operating characteristic (ROC) curves of the PLA for significant fibrosis using the full sample, F2-F4, (FIG. 1A); advanced fibrosis, F3-4, (FIG. 1B); and cirrhosis, F4 (FIG. 1C). Fibrosis staged according to METAVIR.

FIG. 2. Box plots of the PLA according to fibrosis state (N=3797). Fibrosis staged according to METAVIR. Middle line represents median, inferior and superior ends of boxes represent 25th and 75th percentile respectively. Whiskers are 25th and 75th percentile. Dots represent outliers.

FIG. 3. Application of Predictive Liver Assessment Model to the Validation Set (n=3797).

### DETAILED DESCRIPTION OF THE INVENTION

Provided herein are methods of identifying liver fibrosis and monitoring the progression or treatment of liver fibrosis in a patient comprising the steps of:
a) measuring the levels of two biochemical markers (Gamma Glutamyl Transferase (GGT) and Aspartate Aminotransferase (AST) or Alanine Aminotransferase (ALT)) in a sample from the patient,
b) combining the values for each marker, age, and systolic blood pressure, Body Mass Index (BMI), standing height, and weight in an equation that gives a weight to each factor to determine a score,
c) comparing the score to a cut-off value in order to determine the presence or extent of liver fibrosis.

### Samples

As used herein, the term “sample” refers to a biological specimen including serum or plasma taken from the individual to be assessed for liver fibrosis that may contain one or more markers such as GGT, and AST or ALT.

One skilled in the art would understand that the levels of the GGT, and AST or ALT may be assayed in a single sample or may each be assayed from separate samples, provided that the samples are obtained on the same day. The separate samples may be the same type of sample (e.g., serum) or may be of different types (e.g., serum or plasma).

### Determination of Marker Levels

### Gamma Glutamyl Transferase

Gamma glutamyl transferase (GGT), sometimes called γ-glutamyl transpeptidase (GGPT), is an enzyme that is compared with alkaline phosphatase (ALP) levels to distinguish between skeletal disease and liver disease. Because GGT is not increased in bone disorders, as is ALP, a normal GGT with an elevated ALP would indicate bone disease. Conversely, because the GGT is more specifically related to the liver, an elevated GGT with an elevated ALP would strengthen the diagnosis of liver or bile-duct disease.

GGT levels are preferably determined using an automated biochemistry analyzer such as Hitachi 917 biochemistry analyzer (Mannheim, Germany) with Roche Diagnostics reagents. In this method, GGT is measured in fresh serum within 36 hours of collection using this procedure. R1 reagent (123 mmol/L TRIS (i.e., tris(hydroxymethyl)-aminomethane) buffer, pH 8.25 (25° C.); 123 mmol/L glycylglycine; preservative; additive) is added to the sample. R2 reagent (10 mmol/L acetate buffer, pH 4.5 (25° C.); 25 mmol/L L-γ-glutamyl-3-carboxy-4-nitroanilide; stabilizer; preservative) is added to start the formation of L-γ-glutamyl-glycylglycine and 5-amino-2-nitrobenzoate from L-γ-glutamyl-3-carboxy-4-nitroanilide and glycylglycine in the presence of GGT. Gamma-glutamyltransferase transfers the γ-glutamyl group of L-γ-glutamyl-3-carboxy-4-nitroanilide to glycylglycine. The amount of 5-amino-2-nitrobenzoate liberated is proportional to the GGT activity and can be measured photometrically. Samples, controls, and reagents are placed into the analyzer, set up to run according to the manufacturer's protocol, the assay is run, and the results are automatically calculated. The results are reported in U/L, which can be converted to μkat/L by multiplying by a factor of 0.0167.

GGT levels can be determined by other methods known in the art provided such methods produce a result comparable to that obtained with the preferred method.

Aspartate Aminotransferase (AST), also known as Serum Glutamic Oxaloacetic Transaminase (SGOT), is a vital enzyme involved in the interconversion of amino acids and alpha-ketoacids by facilitating the transfer of amino groups. Distributed across various human tissues, including the liver, kidneys, heart, skeletal muscle, adipose tissue, gastric mucosa, brain, and lung tissue, AST plays a crucial role in cellular metabolism. AST is present in both the cytoplasm and mitochondria of cells, with mild tissue damage releasing more of the cytoplasmic form and severe tissue damage releasing more of the mitochondrial form. Elevated circulating AST levels are associated with various diseases, including myocardial infarction, hepatic disease, muscular dystrophy, and organ damage, making AST a valuable marker for assessing tissue health.

Alanine Aminotransferase (ALT), also known as Serum Glutamic Pyruvic Transaminase (SGPT), is a crucial enzyme participating in the conversion of amino acids and alpha-ketoacids by facilitating the transfer of amino groups. Found in various human tissues, including the liver, kidneys, heart, skeletal muscle, and pancreas, ALT plays a pivotal role in cellular metabolism. ALT primarily resides in the cytoplasm of hepatocytes, and its release into the bloodstream is indicative of liver cell damage. Mild liver tissue damage results in a moderate increase in circulating ALT levels, while more severe damage leads to a substantial release of ALT. Thus an elevation of the enzyme activity in serum is a strong indicator of parenchymal liver disease. Elevated ALT levels are associated with a range of conditions, including liver diseases such as hepatitis, cirrhosis, and fatty liver disease.

The determination of AST or ALT activity follows a modification of the method recommended by the International Federation of Clinical Chemistry (IFCC). AST catalyzes the reaction between alpha-ketoglutarate and L-aspartate, forming L-glutamate and oxaloacetate. In a kinetic rate reaction, under the action of malate dehydrogenase (MDH), oxaloacetate converts to malate, and the decrease in absorbance of NADH, measured at 340 nm (secondary wavelength=700 nm), is directly proportional to the serum activity of AST. Similarly, ALT catalyzes the reaction of alpha-ketoglutarate with L-alanine to form L-glutamate and pyruvate. Under the action of LDH, pyruvate converts to lactate, and NADH is converted to NAD. The decrease in absorbance of NADH, measured at 340 nm (secondary wavelength is 700 nm), is directly proportional to the serum activity of ALT.

AST or ALT levels are preferably determined using an automated biochemistry analyzer such as the Roche/Hitachi Cobas 6000 series. This fully automated system is designed for random-access, software-controlled immunoassay, and photometric analyses. The photometric system within the Cobas 6000 can measure colorimetric or immunoturbidimetric reactions using endpoint or kinetic absorbance measurements. The analyzer utilizes a combination of photometric and ion-selective electrode (ISE) determinations, enabling comprehensive in vitro determinations. The results obtained from the Cobas 6000 are reported in standard units per liter (U/L), and the system supports manual or barcode-based bi-directional interface for test ordering, execution, and data entry.

AST or ALT levels may be determined by other methods known in the art provided such methods produce a result comparable to that obtained with the preferred method.

### Determination of a PLA Score

Levels of the markers as determined above, along with diabetes status, age, BMI, systolic blood pressure, height, and weight are input in the following equation to determine a value, y.

$$y = {Lg}_{10} ( systolic blood pressure * BMI ) + ( 0.02 * GGT ) + ( 0.6 * ( AST or ALT ) * diabetes ) + ( 16 * ( weight / standing height ) ) + ( 5 * {Lg}_{10} ( age ) )$$

wherein, systolic blood pressure is in mmHg, BMI=(weight in kg)/(standing height in m) 2, GGT is in (IU/L), diabetes=1 if the individual has diabetes, diabetes=0 if the individual does not have diabetes, AST or ALT is in (U/L), weight is in kilograms (kg), standing height is in centimeters (cm), and age is in years.

### Determination of Presence and Stage of Liver Fibrosis

The end value or score is compared to a cut-off value, in order to identify significant fibrosis (METAVIR stages F2 to F4), advanced fibrosis (stages F3 and F4), or cirrhosis (stage F4). By extension, any value below the cut-off value for significant fibrosis, advanced fibrosis, or cirrhosis indicates an absence of that category of fibrosis.

Significant fibrosis (stages F2 to F4) can be distinguished from an absence of advanced fibrosis (F3 and F4). A score greater than or equal to a cut-off value of about 21.34 (preferably 21.34) is indicative of significant fibrosis, whereas, a score less than a cut-off value of about 21.34 (preferably 21.34) is indicative of an absence of significant fibrosis. A score of greater than or equal to a cut-off value of about 21.63 (preferably 21.63) is indicative of advanced fibrosis, whereas a score of less than a cut-off value of about 21.63 (preferably 21.63) is indicative of the absence of advanced fibrosis. A score of greater than or equal to a cut-off value of about 22.84 (preferably 22.84) is indicative of cirrhosis, whereas a score of less than a cut-off value of about 22.84 (preferably 22.84) is indicative of the absence of cirrhosis.

The following examples serve to illustrate the present invention. These examples are in no way intended to limit the scope of the invention.

### Example 1

Prediction of Fibrosis in Patients with Non-Alcoholic Fatty Liver Disease. In this study, the PLA score was used for the prediction of liver fibrosis among individuals with diagnosed or undiagnosed non-alcoholic fatty liver disease (NAFLD). A retrospective analysis was conducted using cross-sectional, stratified, multistage probability sample data collected by the National Health and Nutrition Examination Survey (NHANES) between 2017 and 2018. The NHANES protocol was approved by the National Center for Health Statistics institutional review board, and written informed consent was obtained from all participants.

Participants completed a home-based interview followed by biosample collection and a physical exam at a mobile examination center (MEC). During the MEC visit, trained technicians conducted physical examinations, including vibration-controlled transient elastography (VCTE). Participants' body mass index (BMI) was calculated using height and weight measured during the physical exam. Variables of interest derived from laboratory tests included albumin, aspartate aminotransferase (AST), alanine aminotransferase (ALT), gamma glutamyl transferase (GGT), platelet count, fasting plasma glucose, and cholesterol. Participants' age and history of diabetes were collected via self-report.

To focus on NAFLD, only participants >20 years of age with valid VCTE test data were included in the analysis. Individuals reporting high alcohol consumption (>30 grams/day for men or >20 grams/day for women) were not included in the analysis (n=235). Additionally, 349 individuals were excluded due to the presence of hepatitis C ribonucleic acid or hepatitis B core antibody, and another 79 were excluded due to self-reported autoimmune hepatitis or other chronic liver condition not including NAFLD.

VCTE was accomplished using FibroScan (Echosens, Paris, France). Pregnant women were not eligible for VCTE. Additionally, those with implanted electronic medical devices and those wearing a bandage or who had lesions where the measurements would be taken were not eligible. Examinations were deemed complete if the participant had fasted at least 3 hours prior to the test and if the technician was able to obtain at least 10 valid stiffness measurements with a liver stiffness interquartile (IQRe) range/median stiffness of less than 30%. All exams were completed using either the M or XL probe. Further detail regarding the FibroScan examination procedure has been previously reported (Gastroenterologie clinique et biologique 32:58-67 (2008)). Participants were categorized by fibrosis stage based on median stiffness measurements. Fibrosis stage F0/F1, indicating no or mild liver scarring were identified by scores of <8.2 kPa, F2 (moderate liver scarring) by 8.2 to 9.6 kPa, and F3/F4 (severe liver scarring) by ≥9.7 kPa (Gastroenterology 156:1717-1730 (2019)).

Individuals were randomly allocated to either the training or validation training set using a 70:30 split. Table 1 details the characteristics of the two groups. Weighted estimates are provided using sample-specific weights. Variances following complex sampling procedures were estimated with Taylor series linearization. Prevalence estimates are reported with their 95% Wald confidence intervals (CI). The overall accuracy of the test was assessed by measuring diagnostic odds ratios (DOR). The DORs were computed to provide a single metric of overall efficacy for each measure as it is independent of disease prevalence while accounting for both sensitivity and specificity (Journal of clinical epidemiology 56:1129-1135 (2003)). In contrast to observed positive predictive value (PPV) and negative predictive value (NPV) the DOR is not as affected by disease prevalence. PPV and NPV are not suitable for low prevalence diseases such as liver fibrosis (Int J cadriovasc sci 29:218-22 (2016)).

TABLE 1
Weighted estimates for clinical and laboratory
features of the training and validation cohorts

| Variable | Training Set | | Validation Set | |
|---|---|---|---|---|
| Weighted sample size, N | 124,639,711 | | 55,608,657 | |
| Unweighted count, n | 2618 | | 1179 | |
| Age, years, mean (95% CI) | 48 | (46-49) | 47 | (45-49) |
| standing height, cm, mean (95% CI) | 168 | (167-169) | 167 | (167-168) |
| weight, kg, mean (95% CI) | 84.7 | (83.0-86.5) | 83.8 | (81.1-86.5) |
| BMI, kg/m2, mean (95% CI) | 30 | (29-31) | 30 | (29-31) |
| Systolic blood pressure, mmHg, mean (95% CI) | 124 | (122-125) | 122 | (120-124) |
| AST, U/L, mean (95% CI) | 21.2 | (20.6-21.8) | 21.6 | (20.9-22.3) |
| GGT, IU/L, mean (95% CI) | 27.5 | (26.0-29.0) | 27.2 | (25.6-28.7) |
| Diabetes, % (95% CI) | 11.5 | (9.9-13.4) | 11.6 | (9.6-14.0) |
| Stage F0-1, % (95% CI) | 91.3 | (89.3-92.9) | 92.0 | (88.9-94.3) |
| Stage F2, % (95% CI) | 3.0 | (2.2-4.1) | 2.7 | (1.4-5.4) |
| Stage F3, % (95% CI) | 2.3 | (1.5-3.5) | 3.1 | (2.3-4.2) |
| Stage F4, % (95% CI) | 3.4 | (2.6-4.5) | 2.2 | (1.4-3.3) |

Univariate and multi variable logistic regression analyses revealed age, systolic blood pressure, AST, GGT, BMI, standing height, weight, and diabetes status to be associated with significant fibrosis. The final predictive model was computed from the results of these factors.

### Example 2

Statistical Analysis. Using the data from the training set, associations between biochemical markers and the presence or absence of advanced fibrosis were assessed using multivariable regression models. In addition, the diagnostic accuracy of each biochemical marker was assessed using receiver operating characteristic (ROC) curve analysis. Markers with significant fibrosis associations were combined with sociodemographic variables and entered into stepwise logistic regression analysis using a backward elimination procedure with a significance level of P=0.10. The dependent variable was defined as advanced fibrosis. Models exhibiting a high AUC or demonstrating a significant level on univariate analysis were combined to form novel multivariable models. These models, built upon various marker combinations, were subsequently assessed through receiver operating characteristic (ROC) curves to ascertain the one most proficient in identifying significant fibrosis accurately. The chosen model, characterized by the fewest variables and the largest area under the curve (AUC), was then employed on the validation set. The resulting regression model was as follows:

$$y = {Lg}_{10} ( systolic blood pressure * BMI ) + ( 0.02 * GGT ) + ( 0.6 * ( AST or ALT ) * diabetes ) + ( 16 * ( weight / standing height ) ) + ( 5 * {Lg}_{10} ( age ) )$$

wherein, systolic blood pressure is in mmHg, BMI=(weight in kg)/(standing height in m) 2, GGT is in (IU/L), diabetes=1 if the individual has diabetes, diabetes=0 if the individual does not have diabetes, AST or ALT is in (U/L), weight is in kilograms (kg), standing height is in centimeters (cm), and age is in years.

Sensitivity, specificity, and the DOR for significant fibrosis, advanced fibrosis and cirrhosis were determined for various cut-off points in the training set and validation set. Clinical and demographic characteristics between the training and validation sets were compared using the Rao-Scott chi-square test for categorical variables and general linear models for continuous variables. All statistical tests were performed using a P<0.05 level of significance. Analyses were conducted using SPSS Complex Samples (SPSS version 25, IBM, Chicago, IL).

### Example 3

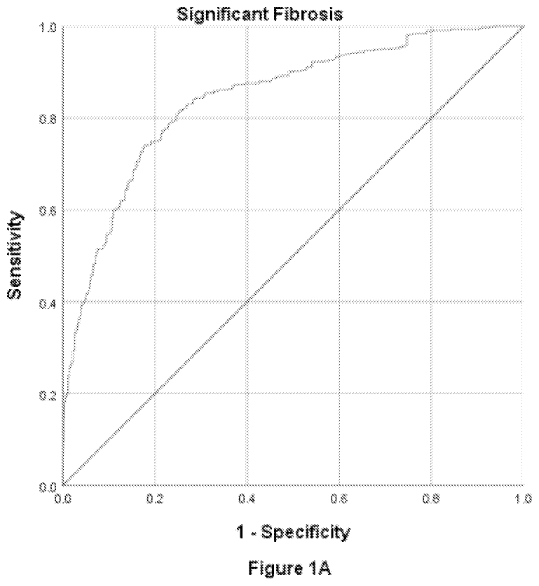

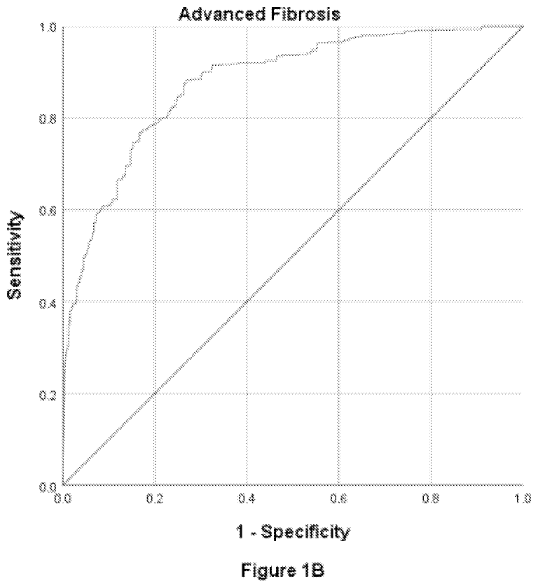

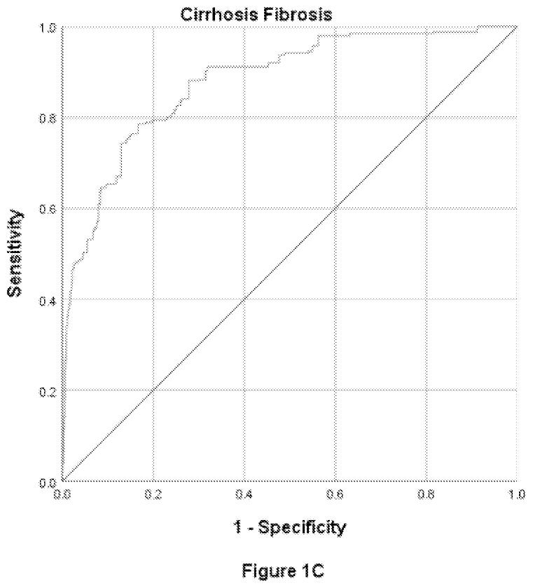

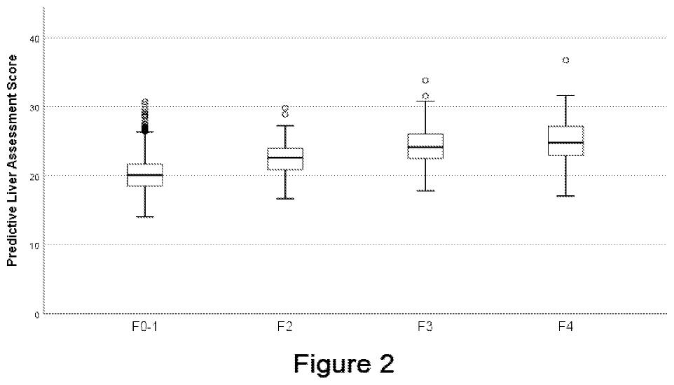

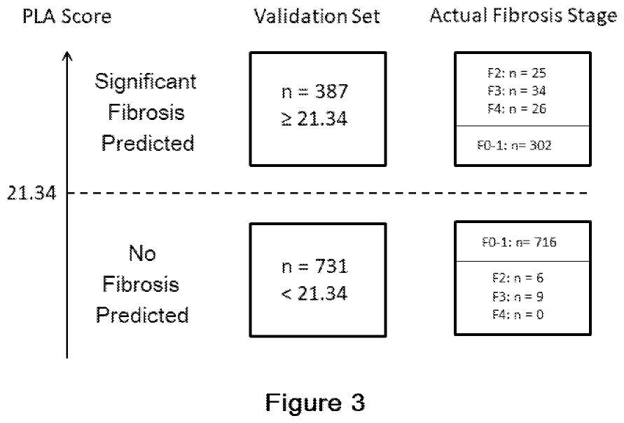

Predictive Model. Biochemical markers assessed in the training set, were combined with sociodemographic factors in logistic regression analysis to create several models which were predictive of significant fibrosis. The optimal multivariable model was considered as having the largest AUC using ROC analysis. This model (PLA) consisted of systolic blood pressure, BMI, diabetes status, AST, GGT, weight, height, and age which provided a high AUC (95% CI) for the prediction of significant fibrosis (0.825 (95% CI, 0.825-0.0825)), as well as for advanced fibrosis (0.867 (95% CI, 0.867-0.0867)) and cirrhosis (0.864 (95% CI, 0.864-0.0864)). AUC graphs for the full dataset are shown in FIG. 1A-C.

A cut-off point of 21.34 among the training set, predicted significant fibrosis (F2 to F4) with a DOR of 10.65. Applying a cut-off point of 21.63 for the prediction of advanced fibrosis (F3 and F4) resulted in a DOR of 18.37. Applying a cut-off point of 22.84 for the prediction of cirrhosis (F4) resulted in a DOR of 16.17.

### Example 4

Model Validation. Using the same cutoff points as for the training data set, the PLA model was applied to the 1179 individuals, statistically weighted to represent 55,608,657 individuals, in the validation set. The consequent AUC was 0.884 (95% CI, 0.884-0.884) for significant fibrosis, 0.902 (95% CI, 0.902-0.902) for advanced fibrosis and 0.933 (95% CI, 0.933-0.933) for cirrhosis.

Among the validation cohort, the cut-off point of 21.34 predicted significant fibrosis (F2 to F4) with a DOR of 27.23. The cut-off point of 21.63 for the prediction of advanced fibrosis (F3 and F4) resulted in a DOR of 26.76. The cut-off point of 22.84 for the prediction of cirrhosis (F4) resulted in a DOR of 30.52.

The effectiveness of a diagnostic test in predicting outcomes is contingent upon the underlying prevalence of the disease. Therefore, given that significant fibrosis typically prompts treatment recommendations (Hepatology 39:1147-71 (2004)), individuals with a PLA score equal to or exceeding 21.34 should be considered for additional testing or therapy, obviating the need for a liver biopsy. Moreover, the exclusion of advanced fibrosis in patients with a PLA score below 21.34 proves particularly valuable for providing prognostic insights, especially for those averse to biopsy, facing significant biopsy-related risks, or elderly patients unlikely to develop liver-related morbidity or mortality without advanced fibrosis (Lancet 349:825-32 (1997)). Lastly, a score at or above 22.84 signals the presence of cirrhosis, offering valuable information to potentially bypass liver biopsy in cases where occult cirrhosis is suspected. This data can guide decisions on variceal and cancer screening, as well as inform patient follow-up strategies (Gut 49:11-21 (2001); WJG 15:2190 (2009)).

Unless expressly defined otherwise, all technical and scientific terms utilized in this document carry the standard meaning understood by individuals possessing ordinary skill in the relevant field to which this invention pertains.

The inventions described herein may suitably be practiced in the absence of any element or elements, limitation or limitations, not specifically disclosed herein. Therefore, terms such as “comprising,” “including,” and “containing” should be interpreted broadly and without limitation. Furthermore, the terms and phrases employed herein have been used as terms of description and not of limitation, and there is no intention in the use of such terms and expressions of excluding any equivalents of the features shown and described or portions thereof. It is acknowledged that various modifications are feasible within the scope of the claimed invention.

Thus, it should be understood that although the present invention has been specifically disclosed through preferred embodiments and optional features, those skilled in the art may make modifications, improvements, and variations to the embodiments presented herein. Such adaptations are considered to be within the scope of this invention. The materials, methods, and examples presented here are indicative of preferred embodiments, serving as illustrations rather than limitations on the breadth of the invention.

The invention has been described broadly and generically herein. Each of the narrower species and subgeneric groupings falling within the generic disclosure also form part of the invention. This includes the general description of the invention with a proviso or negative limitation removing any subject matter from the genus, regardless of whether or not the excised material is specifically recited herein.

In addition, where features or aspects of the invention are described in terms of Markush groups, those skilled in the art will recognize that the invention is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

Other embodiments are set forth within the following claims.

### REFERENCES

1. Younossi Z M, Koenig A B, Abdelatif D, Fazel Y, Henry L, Wymer M. Global epidemiology of nonalcoholic fatty liver disease—meta-analytic assessment of prevalence, incidence, and outcomes. Hepatology. 2016 July; 64(1):73-84.
2. Starley B Q, Calcagno C J, Harrison S A. Nonalcoholic fatty liver disease and hepatocellular carcinoma: a weighty connection. Hepatology. 2010 May; 51(5):1820-32. Hernandez-Gea V, Friedman S L. Pathogenesis of liver fibrosis. Annu Rev Pathol. 2011; 6:425-56.
3. Armandi A, Bugianesi E. Natural history of NASH. Liver International. 2021 June; 41:78-82.
4. Pinzani M. Pathophysiology of liver fibrosis. Digestive diseases. 2015 Jul. 6; 33(4):492-7.
5. Liu Z, Wei X, Chen T, Huang C, Liu H, Wang Y. Characterization of fibrosis changes in chronic hepatitis C patients after virological cure: A systematic review with meta-analysis. Journal of gastroenterology and hepatology. 2017 March; 32(3):548-57.
6. Bedossa P, Poynard T. An algorithm for the grading of activity in chronic hepatitis C. Hepatology. 1996 August; 24(2):289-93.
7. Booth J C, O'Grady J, Neuberger J. Clinical guidelines on the management of hepatitis C. Gut. 2001 Jul. 1; 49(suppl 1):I1-21.
8. Kanwal F, Shubrook J H, Adams L A, Pfotenhauer K, Wong V W, Wright E, Abdelmalek M F, Harrison S A, Loomba R, Mantzoros C S, Bugianesi E. Clinical care pathway for the risk stratification and management of patients with nonalcoholic fatty liver disease. Gastroenterology. 2021 Nov. 1; 161(5):1657-69.
9. Strader D B, Wright T, Thomas D L, Seeff L B. Diagnosis, management, and treatment of hepatitis C. Hepatology. 2004 April; 39(4):1147-71.
10. Kaplan D E, Bosch J, Ripoll C, Thiele M, Fortune B E, Simonetto D A, Garcia-Tsao G. AASLD practice guidance on risk stratification and management of portal hypertension and varices in cirrhosis. Hepatology. 2023 Oct. 23:10-97.
11. Rockey D C, Caldwell S H, Goodman Z D, Nelson R C, Smith A D. Liver biopsy. Hepatology. 2009 Mar. 1; 49(3):1017-44.
12. Sharma S, Khalili K, Nguyen G C. Non-invasive diagnosis of advanced fibrosis and cirrhosis. World journal of gastroenterology: WJG. 2014 Dec. 12; 20(45):16820.
13. Chou R, Wasson N. Blood tests to diagnose fibrosis or cirrhosis in patients with chronic hepatitis C virus infection: a systematic review. Annals of internal medicine. 2013 Jun. 4; 158(11):807-20.
14. van Katwyk S, Coyle D, Cooper C, Pussegoda K, Cameron C, Skidmore B, Brener S, Moher D, Thavorn K. Transient elastography for the diagnosis of liver fibrosis: a systematic review of economic evaluations. Liver international. 2017 June; 37(6):851-61.
15. Staufer K, Halilbasic E, Spindelboeck W, Eilenberg M, Prager G, Stadlbauer V, Posch A, Munda P, Marculescu R, Obermayer-Pietsch B, Stift J. Evaluation and comparison of six noninvasive tests for prediction of significant or advanced fibrosis in nonalcoholic fatty liver disease. United European gastroenterology journal. 2019 October; 7(8):1113-23.
16. Papatheodoridi M, Cholongitas E. Diagnosis of non-alcoholic fatty liver disease (NAFLD): current concepts. Current pharmaceutical design. 2018 Oct. 1; 24(38):4574-86.
17. Morling J R, Fallowfield J A, Guha I N, Nee L D, Glancy S, Williamson R M, Robertson C M, Strachan M W, Price J F, Edinburgh Type 2 Diabetes Study investigators. Using non-invasive biomarkers to identify hepatic fibrosis in people with type 2 diabetes mellitus: the Edinburgh type 2 diabetes study. Journal of hepatology. 2014 Feb. 1; 60(2):384-91.
18. Cheng J, Hou J, Ding H, Chen G, Xie Q, Wang Y, Zeng M, Ou X, Ma H, Jia J. Validation of ten noninvasive diagnostic models for prediction of liver fibrosis in patients with chronic hepatitis B. PLoS One. 2015 Dec. 28; 10(12):e0144425.
19. De Ledinghen V, Vergniol J. Transient elastography (fibroscan). Gastroenterologie clinique et biologique. 2008 Sep. 1; 32(6):58-67.
20. Eddowes P J, Sasso M, Allison M, Tsochatzis E, Anstee Q M, Sheridan D, Guha I N, Cobbold J F, Deeks J J, Paradis V, Bedossa P. Accuracy of FibroScan controlled attenuation parameter and liver stiffness measurement in assessing steatosis and fibrosis in patients with nonalcoholic fatty liver disease. Gastroenterology. 2019 May 1; 156(6):1717-30.
21. Glas A S, Lijmer J G, Prins M H, Bonsel G J, Bossuyt P M. The diagnostic odds ratio: a single indicator of test performance. Journal of clinical epidemiology. 2003 Nov. 1; 56(11):1129-35.
22. Borges L S. Diagnostic accuracy measures in cardiovascular research. Int J Cardiovasc Sci. 2016 September; 29(3):218-22.
23. Poynard T, Bedossa P, Opolon P. Natural history of liver fibrosis progression in patients with chronic hepatitis C. The Lancet. 1997 Mar. 22; 349(9055):825-32.
24. Sebastiani G. Non-invasive assessment of liver fibrosis in chronic liver diseases: implementation in clinical practice and decisional algorithms. World journal of gastroenterology: WJG. 2009 May 5; 15(18):2190.

1. A method of identifying liver fibrosis in an individual person, said method comprising:
a) assaying the concentration of gamma glutamyl transferase (GGT) and aspartate aminotransferase (AST) or alanine aminotransferase (ALT) in a bodily sample from said person;
b) determining the height, weight, BMI, systolic blood pressure, age, and diabetes status of said person;
c) determining an end value (y) using an algorithm, wherein said algorithm is,
$$y = {Lg}_{10} ( systolic blood pressure * BMI ) + ( {C}_{1} * GGT ) + ( {C}_{2} * ( AST or ALT ) * diabetes ) + ( {C}_{3} * ( weight / standing height ) ) + ( {C}_{4} * {Lg}_{10} ( age ) )$$
wherein,
C₁ is between 0.001 and 0.02 inclusive,
C₂ is between 0.01 and 0.5 inclusive,
C₃ is between 5 and 50 inclusive,
C₄ is between 0.1 and 15 inclusive, and
wherein,
age is in years,
systolic blood pressure is in mmHg,
presence of diabetes=1, lack of diabetes=0,
BMI is in kg/m²,
GGT is in IU/L,
AST or ALT is in U/L,
weight (kg)
standing height (cm); and
c) comparing said end value, y, to a cut-off value associated with the presence of liver fibrosis and classifying said person with liver fibrosis when the end value is greater than or equal to the cut-off value.

2. The method of claim 1, wherein
$$y = {Lg}_{10} ( systolic blood pressure * BMI ) + ( 0.02 * GGT ) + ( 0.6 * ( AST or ALT ) * diabetes ) + ( 16 * ( weight / standing height ) ) + ( 5 * {Lg}_{10} ( age ) ) .$$

3. The method of claim 1, wherein said cut-off value is 21.34, and wherein an end value, y, of greater than or equal to 21.34 is indicative of significant fibrosis or an end value, y, of less than 21.63 is indicative of the absence of advanced fibrosis.

4. The method of claim 1, wherein said cut-off value is 22.84 and an end value, y, less than 22.84 is indicative of the absence of cirrhosis of the liver.

5. The method of claim 1, wherein said cut-off value is 22.84 and an end value, H, greater than or equal to 22.84 is indicative of the presence of cirrhosis.

6. The method of claim 1, wherein said sample is serum or plasma.

7. The method of claim 1, wherein the end value (y) is employed in determining an appropriate course of treatment for the patient.

8. The method of claim 1, wherein said person suffers from a disease involving liver fibrosis.

9. A method of monitoring progression of liver fibrosis in a patient, said method comprising:
a) assaying the concentration of gamma glutamyl transferase (GGT) and aspartate aminotransferase (AST) or alanine aminotransferase (ALT) in an first bodily sample from said person;
b) determining the height, weight, BMI, systolic blood pressure, age, and diabetes status of said person;
c) determining an end value (y) using an algorithm, wherein said algorithm is,
$$y = {Lg}_{10} ( systolic blood pressure * BMI ) + ( {C}_{1} * GGT ) + ( {C}_{2} * ( AST or ALT ) * diabetes ) + ( {C}_{3} * ( weight / standing height ) ) + ( {C}_{4} * {Lg}_{10} ( age ) )$$
wherein,
C₁ is between 0.001 and 0.02 inclusive,
C₂ is between 0.01 and 0.5 inclusive,
C₃ is between 5 and 50 inclusive,
C₄ is between 0.1 and 15 inclusive, and
wherein,
age is in years,
systolic blood pressure is in mmHg,
presence of diabetes=1, lack of diabetes=0,
BMI is in kg/m²,
GGT is in IU/L,
AST or ALT is in U/L,
weight (kg)
standing height (cm); and
d) comparing said end value (y) to a cut-off value associated with the presence of liver fibrosis, and determining the extent of liver fibrosis in said patient when the end value is greater than or equal to the cut-off value;
e) obtaining a second sample from said patient, wherein said second sample is obtained after said first sample;
f) repeating steps b)-d) to determine the extent of liver fibrosis as indicated by said second sample; and
g) determining progression of liver fibrosis by comparing the extent of liver fibrosis indicated by said first sample to the extent of liver fibrosis indicated by said second sample; wherein a higher extent in liver fibrosis as indicated by the second sample in comparison to the first sample indicates progression in liver fibrosis or a lesser extent in liver fibrosis as indicated by the second sample in comparison to the first sample indicates a regression in liver fibrosis.

10. The method of claim 9, wherein
$$y = {Lg}_{10} ( systolic blood pressure * BMI ) + ( 0.02 * GGT ) + ( 0.6 * ( AST or ALT ) * diabetes ) + ( 16 * ( weight / standing height ) ) + ( 5 * {Lg}_{10} ( age ) ) .$$

11. The method of claim 9, wherein said cut-off value is 21.34, and wherein an end value, y of greater than or equal to said cut-off value is indicative of significant fibrosis or an end value, y of less than said cut-off value is indicative of the absence of advanced fibrosis.

12. The method of claim 9, wherein said cut-off value is 22.84 and an end value, y, of less than 22.84 is indicative of an absence of cirrhosis of the liver.

13. The method of claim 9, wherein said cut-off value is 22.84 and an end value, y, greater than or equal to 22.84 is indicative of the presence of cirrhosis of the liver.

14. The method of claim 9, wherein said sample is serum or plasma.

15. A method of monitoring the efficacy of liver fibrosis therapy in a patient in need thereof, said method comprising:
a) assaying the concentration of gamma glutamyl transferase (GGT) and aspartate aminotransferase (AST) or alanine aminotransferase (ALT) in an first bodily sample from said person;
b) determining the height, weight, BMI, systolic blood pressure, age, and diabetes status of said person;
c) determining an end value (y) using an algorithm, wherein said algorithm is,
$$y = {Lg}_{10} ( systolic blood pressure * BMI ) + ( {C}_{1} * GGT ) + ( {C}_{2} * ( AST or ALT ) * diabetes ) + ( {C}_{3} * ( weight / standing height ) ) + ( {C}_{4} * {Lg}_{10} ( age ) )$$
wherein,
C₁ is between 0.001 and 0.02 inclusive,
C₂ is between 0.01 and 0.5 inclusive,
C₃ is between 5 and 50 inclusive,
C₄ is between 0.1 and 15 inclusive, and
wherein,
age is in years,
systolic blood pressure is in mmHg,
presence of diabetes=1, lack of diabetes=0,
BMI is in kg/m²,
GGT is in IU/L,
AST or ALT is in U/L,
weight (kg)
standing height (cm); and
d) comparing said end value, y, to a cut-off value associated with the presence of liver fibrosis, and determining the extent of liver fibrosis in said patient when the end value is greater than or equal to the cut-off value;
e) obtaining a second sample from said patient, wherein said second sample is obtained after said first sample;
f) repeating steps b)-d) to determine the extent of liver fibrosis as indicated by said second sample; and
g) determining the efficacy of the liver fibrosis therapy by comparing the extent of liver fibrosis indicated by said first sample to the extent of liver fibrosis indicated by said second sample; wherein an equivalent or higher extent in liver fibrosis as indicated by the second sample in comparison to the first sample indicates the liver fibrosis therapy is not efficacious in treating liver fibrosis, or a lesser extent in liver fibrosis as indicated by the second sample in comparison to the first sample indicates the liver fibrosis therapy is efficacious in treating liver fibrosis.

16. The method of claim 15, wherein
$$y = {Lg}_{10} ( systolic blood pressure * BMI ) + ( 0.02 * GGT ) + ( 0.6 * ( AST or ALT ) * diabetes ) + ( 16 * ( weight / standing height ) ) + ( 5 * {Lg}_{10} ( age ) ) .$$

17. The method of claim 15, further comprising predicting said patient has significant or advanced fibrosis when said determined end value, y, is greater than a cut-off value of 21.34, or predicting said patient does not have advanced fibrosis when said determined end value, y, is lower than a cut-off value of 21.63.

18. The method of claim 15, wherein said cut-off value is 22.84 and an end value, y, of less than 22.84 is indicative of an absence of cirrhosis of the liver.

19. The method of claim 15, wherein said cut-off value is 22.84 and an end value, H, greater than or equal to 22.84 is indicative of the presence of cirrhosis of the liver.

20. The method of claim 15, wherein said sample is serum or plasma.
